(19) United States
(12) Patent Application Publication
(10) Pub. No.: US 2012/0017914 A1
(43) Pub. Date: Jan. 26, 2012

(54) ENDOBRONCHIAL TUBE ASSEMBLIES AND SELECTORS

(75) Inventor: Pasi Pun Entaban Anak Watt Lanyau, London (GB)

(73) Assignee: Smiths Medical International Limited, Kent (GB)

(21) Appl. No.: 12/998,574

(22) PCT Filed: Nov. 13, 2009
(86) PCT No.: PCT/GB2009/002649

§ 371 Date: Aug. 4, 2011

(30) Foreign Application Priority Data
Nov. 28, 2008 (GB) 0821739.0

(51) Int. Cl. A61M 16/04

(52) U.S. Cl. 128/207.14

(57) ABSTRACT

An endobronchial tube assembly for selective ventilation of the left or right lung has an endobronchial tube (1) with two passages opening into the left and right lungs. A selector (2) mounted at the machine end (20) of the tube (1) has a rotatable plate (70) with apertures (73, 74, 75) which can be positioned to allow gas to flow along a selected passage of the tube (1). A lock mechanism (90, 81, 82, 83) retains the plate (70) in the selected position until a button (61) is depressed to release the plate, which can then be moved to one side or the other by displacing a knob (77).

This invention relates to endobronchial tube assemblies of the kind including a shaft having two passageways opening towards the patient end of the assembly and seals located towards the patient end of the assembly for sealing the shaft with portions of the respiratory tract such that one passageway opens into one lung and the other passageway opens into the other lung, the assembly including towards its machine end a manually-displaceable selector that can be set in a first state in which the passageway opening into the left lung is open and the passageway opening into the right lung is closed or a second state in which the passageway into the right lung is open and the passageway into the left lung is closed.

Various tube assemblies have been proposed for selective ventilation or anaesthesia of an individual lung. Gale and Waters proposed in 1931 a tube with a single cuff inserted into the right bronchus, the cuff being inflated just above the carina so that it blocks the right bronchus. This had the disadvantage that the cuff could also block the left bronchus and thereby prevent the left lung from collapsing. If the tube were inserted further into the bronchus there would be the danger that the cuff would occlude the upper lobe of the right bronchus. In 1936 Magill proposed a tube assembly with a separate blocker. The tube is positioned so that it opens above the carina and is sealed from the trachea by a cuff. The blocker is in the form of a narrow tube with an inflatable cuff inserted through the main tube into the left bronchus. When inflated, the blocker cuff seals the left bronchus from the right bronchus whereas the bore through the narrow tube enables the left lung to be separately ventilated. Green and Gordon (Anaesthesia Vol 12, No 1, January 1957) described an assembly of a tube with two cuffs. The patient end of the tube is located just below the upper lobe in the right bronchus, with one cuff being inflated to form a seal with the trachea above the carina and the other cuff forming a seal with the right bronchus in the region of the upper lobe, so as to seal the right bronchus from the left bronchus. Conventional endobronchial tubes sold today follow this general configuration. Examples of such tube assemblies are described in GB2168256 and in U.S. Pat. No. 5,309,906, which describe an endobronchial tube sold by Smiths Medical under the Portex Blue Line trade mark (Portex and Blue Line are registered trade marks of Smiths Medical).

Where only one lung is to be ventilated using a conventional endobronchial tube, it is usual practice to close the passage leading to the other lung by means of a clamp, such as forceps, that squeezes together opposite sides of a tube forming a part of that passage at the machine end of the assembly. This has obvious disadvantages. The clamped tube may not open fully without manipulation when the clamp is released. The wrong tube or both tubes could be clamped inadvertently. The seal provided by the clamp may not be completely effective. Also, it is a disadvantage to have to use an additional, separate component in order to close off one lung. WO2007/141487 proposes a rotatable switch device that can be manually displaced to open a selected one of the passageways. U.S. Pat. No. 5,392,772 describes another rotatable device for selecting between lumens in an endotracheal tube. U.S. Pat. No. 4,489,721 describes a double lumen adaptor with two rotatable valve plugs. WO99/19013 describes a double lumen adaptor that is rotatable.

It is very important for such selectors to be easy to operate reliably, that they be resistant to inadvertent change in selected lung, such as by catching on adjacent equipment, bedding or the like, and that the lung that is selected be readily apparent from looking at the selector.

It is an object of the present invention to provide an alternative endobronchial tube assembly and selector.

According to one aspect of the present invention there is provided an endobronchial tube assembly of the above-specified kind, characterised in that the selector includes a rotatable plate with a manually-displaceable member or portion by which the plate can be displaced and a separate lock mechanism arranged to retain the plate it in its selected position, such that the plate can only be displaced by actuating both the lock mechanism and the manually-displaceable member at the same time.

The selector preferably has an outer housing and the rotatable plate is contained within the housing with the manually-displaceable member actuable externally of the housing. The selector preferably has a third state in which the passageways into both lungs are open. The rotatable plate preferably has three holes that align with different ones of two ports in the housing depending on the orientation of the plate. The plate may be of a relatively rigid material and have a sealing layer of a softer material on a surface of the plate. The lock mechanism preferably includes a push button, which may be accessible on a front surface of the selector, the manually-displaceable member being accessible at an upper edge of the selector. The lock mechanism preferably includes a spring member arranged to engage surface formations on the rotatable plate to retain the plate in different orientations. The manually-displaceable member is preferably displaced to the left to open the passageway into the left lung and to the right to open the passageway into the right lung. The manually-displaceable member preferably has a position between the left and right positions in which the passageways into both lungs are open.

According to another aspect of the present invention there is provided a manually-displaceable selector for an endobronchial tube assembly according to the above one aspect of the present invention.

An endobronchial tube assembly and selector according to the present invention will now be described, by way of example, with reference to the accompanying drawings, in which:

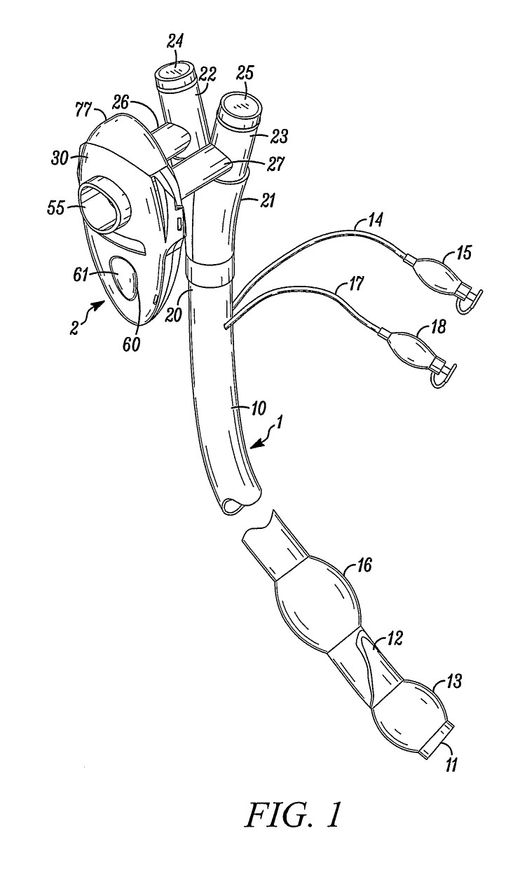

FIG. 1 is a perspective view of the assembly;

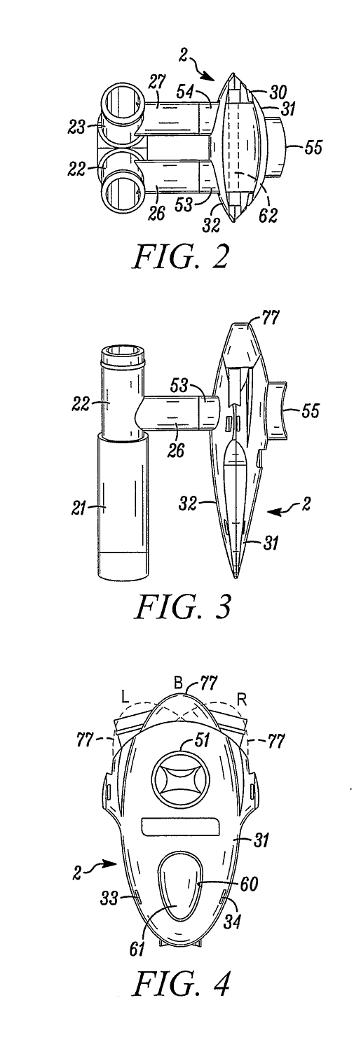

FIG. 2 is a view of the selector from above;

FIG. 3 is a side elevation view of the selector;

FIG. 4 is front view of the selector showing its different settings;

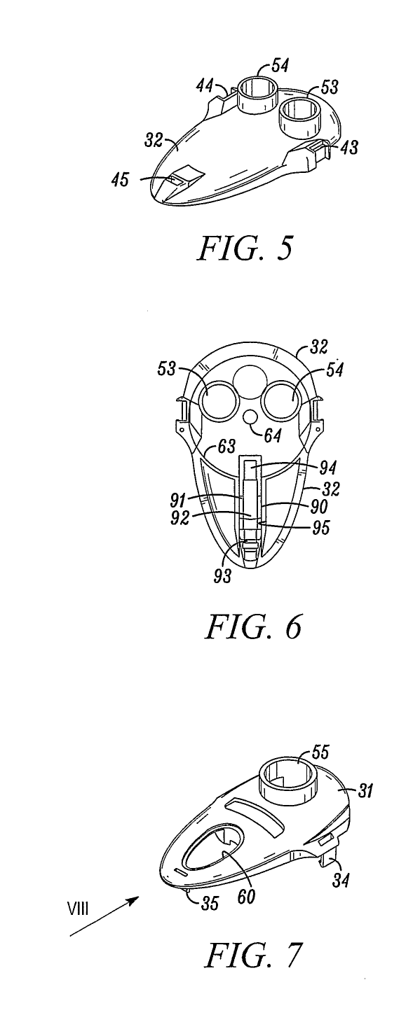

FIG. 5 is a perspective view of the outside of the rear panel of the selector housing;

FIG. 6 is a elevation view of the rear panel of the selector housing with the locking element;

FIG. 7 is a perspective view of the front panel of the housing;

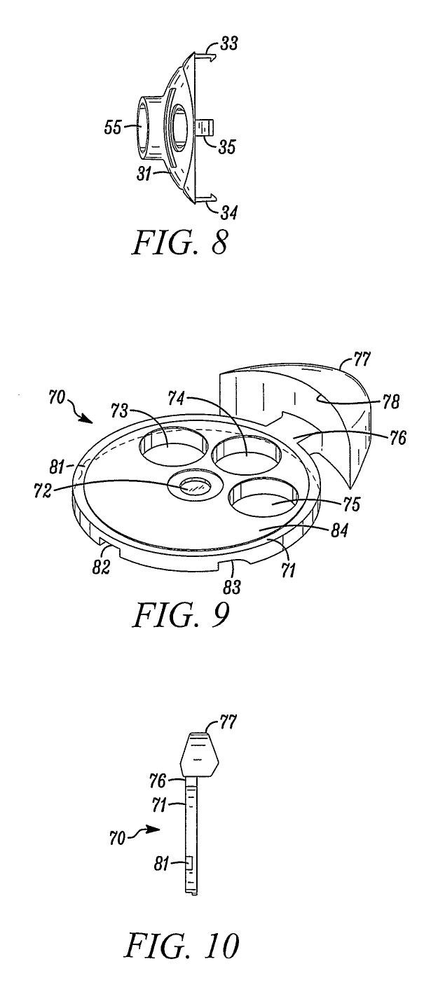

FIG. 8 is a view of the front panel along the arrow VIII of FIG. 7;

FIG. 9 is a perspective view of the rear of the selector plate; and

FIG. 10 is a side view of the selector plate.

With reference first to FIG. 1, the endobronchial assembly comprises an endobronchial tube 1 and a switch or selector 2 at its machine end by which ventilation connection can be made to the tube.

The tube 1 is conventional in having a curved shaft 10 with two gas passageways or lumens extending side-by-side along its length. One passageway opens at the patient end tip 11 of the tube 1, the other passageway opening through a side opening 12 displaced a short distance to the rear of the tip. A first seal in the form of an inflatable cuff 13 encompasses the tube 1 between the patient end tip 11 and the side opening 12, the cuff being inflatable via an inflation line 14 and a combined connector and pilot balloon 15. A second seal cuff 16 is located immediately to the rear of the side opening 12 and is inflated via a second inflation line 17 and a combined connector and pilot balloon 18. When correctly inserted, the patient end tip 11 locates in the right lung and the side opening 12 opens just above the carina so that the left lung can be ventilated. The lower sealing cuff 13 serves to isolate the two lungs from one another while the upper cuff 16 seals the outside of the tube 1 with the lower end of the trachea in the usual way.

The upper, machine end 20 of the shaft 10 connects with a coupling assembly 21 including two branched tubes 22 and 23 connected with respective ones of the passageways extending along the shaft. The tubes 22 and 23 extend at an angle to one another to form a V-shape and are closed at their machine ends by respective caps 24 and 25. Each tube 22 and 23 has a short, laterally-extending side arm 26 and 27 projecting parallel to one another at right angles towards the outside of the curve of the shaft 10. The two side arms 26 and 27 connect, at their outer end with the rear of the selector 2.

With reference now also to the other drawings, the selector 2 has an outer housing 30 of generally oval or tear-drop shape, being broader and more rounded at its upper end and more tapered and narrower at its lower end. Typically, the selector is about 68 mm long (including its knob 77), about 37 mm wide at its broadest and with a depth or thickness of about 14 mm (not including its ports 53, 54 and 55). The axis of the housing 30 is arranged parallel with that of the tube 1, with the narrower end of the housing towards the patient end of the tube. The housing 30 comprises a front and rear plastics moulded panel 31 and 32. The panels 31 and 32 are held together by three snap-fit clips 33, 34 and 35 (shown most clearly in FIGS. 7 and 8) projecting rearwardly from the front panel 31, which project into and lock within respective slots 43 to 45 on the rear panel 32 (shown most clearly in FIGS. 5 and 6). The rear panel 32 is moulded with two short ports 53 and 54 projecting outwardly, side-by-side, to which respective ones of the side arms 26 and 27 are attached by an interference, push-fit or by bonding. The front panel 31 has a single short inlet/outlet port 55 projecting outwardly and positioned midway between the ports 53 and 54 on the opposite side of the housing 30. This port 55 is either left open or is connected via a standard luer connector to a ventilation circuit (not shown).

A second opening 60 of oval or tear-drop shape is located directly below the inlet/outlet port 55 on the front panel 31. The opening 60 receives a push button 61 forming a part of a lock mechanism and accessible on the front surface of the selector 2, which is described in greater detail below.

Inside the housing 30, the rear panel 32 is moulded with an internal circular recess 63 (FIG. 6) occupying most of the width of the upper part of the panel. A short axle or stud 64 projects forwardly centrally within the recess 63. The recess 63 receives a separate selector plate 70 shown in detail in FIGS. 9 and 10. The plate 70 is a single-piece integral moulding of a relatively rigid plastics material and comprises a circular disc portion 71 of the same diameter as the recess 63 and having a small central hole 72 the same diameter as the stud 64. The disc 71 is also formed with three larger holes 73, 74 and 75 of the substantially the same diameter as the ports 53 and 54 in the rear panel 32. The diameter of the holes 73, 74 and 75 could be varied to alter the performance of the device. The holes 73 to 75 are located on an arc concentric with the central hole 72. A short radial extension 76 projects from the upper side of the selector plate 70 and this supports a generally triangular slider knob 77 of increased thickness, which provides a manually-displaceable member of the plate. The lower edge 78 of the knob 77 is curved to follow the external contour of the upper edge of the housing 30. The lower edge of the disc 71 is interrupted by surface formations in the form of three small semicircular notches 81, 82 and 83. The middle notch 82 is located diametrically opposite the extension 76 and the middle hole 74. The other notches 81 and 83 are spaced from the central notch by 60°, diametrically opposite the holes 75 and 73 respectively. The notches 81 to 83 serve a locking function, which is described in detail below. Attached to the rear surface of the disc portion 71 is a softer sealing layer 84, which faces the ports 53 and 54. The sealing layer 84 contacts the panel 32 around the ports so as to seal the plate 70 with the ports and prevent leakage. The sealing layer 84 may be of a deformable, rubber-like material. The selector plate 70 is located in the circular recess 63 within the housing 30 with the central hole 72 in the plate receiving the stud 64. The extension 76 projects through an arcuate slot 62 (FIG. 2) formed between the two housing panels 31 and 32 at the top of the housing 30 so that the knob 77 is located externally of the housing.

The knob 77 is used to rotate the selector plate 70 between three different positions or orientations labelled “B”, “L” and “R” in FIG. 4. In position “B” shown in FIG. 1, the selector plate 70 has a central orientation and the two holes 73 and 75 align with respective ones of the ports 53 and 54 so that gas can flow to or from both lumens of the tube 1 via the side arms 26 and 27, the holes in the plate and through the inlet/outlet port 55 on the front of the selector 2. If, however, the plate 70 were rotated through 60° anticlockwise, when viewed from the front, to its full extent, to position “L”, the middle hole 74 would become aligned with the left port 53 and access to the other port 54 would be blocked by an imperforate region of the plate 70. In this position, gas flowing from the inlet/outlet port 55 can flow only to and from the left lung. Similarly, rotating the plate 70 to its maximum extent in the opposite direction, to position “R” would bring the middle hole 74 into alignment with the right port 54 to isolate the left lung and enable ventilation of the right lung only.

The lower part of the housing 30 contains a locking element in the form of a spring metal strip 90 of rectangular shape, as shown in FIG. 6. The strip 90 is cut to form an outer frame 91 and an integral inner strip 92 attached with the frame at its lower end 93 and unattached at its upper end 94. The inner strip 92 is bent to take a form where it bends forwardly at its upper end 94 away from the upper end of the outer frame 91. The locking element 90 is located in a rectangular recess 95 in the rear panel 32 and projects axially from the lower end of the housing 30 a short distance within the circular recess 63 and beneath the selector plate 70. The dimensions of the inner strip 92 are such that its upper end 94 locates in the middle notch 82 when the plate 70 is in a central “B” orientation. The engagement of the locking element 90 in the notch 82 prevents the plate 70 being rotated. The lower end 93 of the locking element 90 is engaged by an inner surface of the push button 61 mounted in the lower opening 60 in the front panel 31. The button 61 is urged outwardly by the resilience of the inner strip 92 but can be pushed in against this resilience when the selector 2 is to be unlocked. Pushing in the button 61 causes the upper end 94 of the inner strip 92 to be deflected rearwardly below the selector plate 70 and out of engagement with the notch 82 so that the plate is freed to be rotated by manually engaging the knob 77 and sliding it to the left or right. When the button 61 is released, the inner strip 92 is urged against the rear surface of the plate 70 and snaps into the notch 81 or 83 when the plate is oriented such that the notch is aligned with the inner strip.

The normal state of the selector 2 is with the knob 77 in a central “B” orientation and with the inlet/outlet port 55 opening into both lumens of the tube 1 so that both lungs are ventilated. When the clinician needs to isolate one lung he holds the selector 2 in one hand, presses in the push button 61 with his thumb and displaces the knob 77 with the index finger of the same hand to the left or right depending on whether the left or right lung is to be ventilated. When fully displaced to the left or right the selector plate 70 locks in position and cannot be released without actuating the push button 61.

It can be seen that the lock feature of the present arrangement ensures that the setting of the selector 2 cannot be inadvertently changed. Different physical actions on different components of the selector 2 are needed to unlock (pushing in the button 61) and change the setting (angular displacement of the knob 77), thereby enhancing the security of the device. It is readily apparent from observation of the position of the knob 77 which lung is being ventilated, thereby ensuring a very low risk of inappropriate ventilation. To enhance this, the housing 30 could be marked appropriately to indicate which lung was being ventilated at the different positions of the knob 77. The arrangement of the selector has ergonomic benefit in that it can easily be operated using only one hand.

The arrangement of the present invention can be made at low cost, it can be very compact and of low weight to reduce loading on the patient. The selector 2 enables either or both lungs to be ventilated. The construction of the selector 2 is such that there is no intermediate position of the selector plate 70 in which both lungs can be blocked at the same time.

1-11. (canceled)

12. An endobronchial tube assembly including a shaft having two passageways opening towards the patient end of the assembly and seals located towards the patient end of the assembly for sealing the shaft with portions of the respiratory tract such that one passageway opens into one lung and the other passageway opens into the other lung, the assembly including towards its machine end a manually-displaceable selector that can be set in a first state in which the passageway opening into the left lung is open and the passageway opening into the right lung is closed or a second state in which the passageway into the right lung is open and the passageway into the left lung is closed, characterized in that the selector includes a rotatable plate with a manually-displaceable member or portion by which the plate can be displaced and a separate lock mechanism arranged to retain the plate in its selected position, such that the plate can only be displaced by actuating both the lock mechanism and the manually-displaceable member at the same time.

13. An assembly according to claim 12, characterized in that the selector includes an outer housing and that the rotatable plate is contained with the housing with the manually-displaceable member being actuable externally of the housing.

14. An assembly according to claim 12, characterized in that the selector has a third state in which the passageways into both lungs are open.

15. An assembly according to claim 12, characterized in that the rotatable plate has three holes that align with different ones of two ports in the housing depending on the orientation of the plate.

16. An assembly according to claim 12, characterized in that the plate is of a relatively rigid material and has a sealing layer of a softer material on a surface of the plate.

17. An assembly according to claim 12, characterized in that the lock mechanism includes a push button.

18. An assembly according to claim 17, characterized in that the push button is accessible on a front surface of the selector and the manually-displaceable member is accessible at an upper edge of the selector.

19. An assembly according to claim 12, characterized in that the lock mechanism includes a spring member arranged to engage surface formations on the rotatable plate to retain the plate in different orientations.

20. An assembly according to claim 12, characterized in that the manually-displaceable member is displaced to the left to open the passageway into the left lung and to the right to open the passageway into the right lung.

21. An assembly according to claim 20, characterized in that the manually-displaceable member has a position between the left and right positions in which the passageways into both lungs are open.

22. A manually-displaceable selector for an endobronchial tube assembly according to claim 12.
